(19) United States
(12) Patent Application Publication
(10) Pub. No.: US 2010/0210681 A1
(43) Pub. Date: Aug. 19, 2010

(54) AQUEOUS PHARMACEUTICAL COMPOSITION

(75) Inventors: Holger Bauer, Heidelberg (DE); Norbert Rasenack, Weil am Rhein (DE); Kerstin Ammer, Darmstadt (DE); Bianka Kemmerer, Seligenstadt (DE); Kirstin Heil, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: MERCK PATENT GESELLSCHAFT MIT BESCHRÄNKTER HAFTUNG, Darmstadt (DE)

(21) Appl. No.: 12/599,427

(22) PCT Filed: Apr. 24, 2008
(86) PCT No.: PCT/EP2008/003291

§ 371 Date: Nov. 9, 2009

(30) Foreign Application Priority Data
May 10, 2007 (DE) 10 2007 021 862.3

(51) Int. Cl. A61K 31/436; A61P 35/00

(52) U.S. Cl. 514/302

(57) ABSTRACT

The present invention relates to an aqueous pharmaceutical composition comprising 1-(2-dimethylaminoethyl)-3-((2R,4aS,5R,10bS)-5-phenyl-9-trifluoromethyl-3,4,4a,5,6,10b-hexahydro-2H-pyrano[3,2-c]quinolin-2-ylmethyl)urea as active compound, to a liquid pharmaceutical composition which is suitable for the preparation of the aqueous pharmaceutical composition, and to a process for the preparation of the compositions.

The present invention relates to an aqueous pharmaceutical composition comprising 1-(2-dimethylaminoethyl)-3-((2R,4aS,5R,10bS)-5-phenyl-9-trifluoromethyl-3,4,4a,5,6,10b-hexahydro-2H-pyrano[3,2-c]quinolin-2-yl-methyl)urea as active compound, to a liquid pharmaceutical composition which is suitable for the preparation of the aqueous pharmaceutical composition, and to a process for the preparation of the compositions.

1-(2-Dimethylaminoethyl)-3-((2R,4aS,5R,10bS)-5-phenyl-9-trifluoromethyl-3,4,4a,5,6,10b-hexahydro-2H-pyrano[3,2-c]quinolin-2-ylmethyl)urea is a specific inhibitor of the mitotic motor protein Eg5 of the following structural formula:

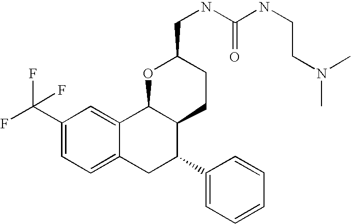

Inhibition of the motor protein Eg5 results in collapse of the spindle fibres, with the consequence that the chromosomes are no longer divided correctly amongst the daughter cells during cell division. Overall, this results in mitotic arrest and thus in the dividing cells dying off. Owing to the specific function of Eg5 during cell division (mitosis), the action relates principally to fast-dividing and incompletely differentiated cells.

1-(2-Dimethylaminoethyl)-3-((2R,4aS,5R,10bS)-5-phenyl-9-trifluoromethyl-3,4,4a,5,6,10b-hexahydro-2H-pyrano[3,2-c]quinolin-2-ylmethyl)urea is a highly promising active compound for the treatment of various diseases and complaints which are associated with increased angiogenesis, such as cancer, tumour formation, growth and spread, arteriosclerosis, ocular diseases, choroidal neovascularisation and diabetic retinopathy, inflammatory diseases, arthritis, neurodegeneration, restenosis, wound healing or trans-plant rejection. The use of this active compound is of particular interest for the therapy or prophylaxis of cancer diseases.

Above and below, “active compound” means 1-(2-dimethylaminoethyl)-3-((2R,4aS,5R,10bS)-5-phenyl-9-trifluoromethyl-3,4,4a,5,6,10b-hexahydro-2H-pyrano[3,2-c]quinolin-2-ylmethyl)urea.

If the active compound is employed in intensive medicine, such as, for example, in the treatment of cancer diseases, it can preferably be administered parenterally as a solution. For therapeutic use, solutions of the active compound which can be administered parenterally, in particular aqueous solutions, are therefore desirable. Suitable aqueous solutions of the active compound should be matched to the particular therapy requirements, in particular they should comprise the active compound in the therapeutically necessary amount and have adequate storage stability.

However, attempts to prepare aqueous solutions of the active compound which can be administered parenterally have shown that the compound is extremely unstable. Thus, the active compound is so strongly degraded after just a few hours of storage at room temperature in aqueous solution, for example an active-compound solution in physiological saline solution (approximately 20% degradation after 2 hours), that it is no longer suitable for therapeutic administration to humans.

Owing to the rapid degradation, reproducible administration of the active compound as an aqueous solution in the amount and quality necessary in each case cannot be ensured in practice. Furthermore, the active-compound degradation which takes place in aqueous solution is accompanied by an increase in degradation products, which are undesired from a toxicological point of view. Owing to the high active-compound degradation rate, these problems likewise cannot be avoided by preparing the aqueous active-compound solution just before administration thereof. This method would in addition also have the disadvantage that it would be accompanied by significantly increased logistical complexity and would therefore be very difficult to implement in routine clinical procedures. The instability of the active compound in aqueous solution thus puts parenteral administration of aqueous solutions of the active compound in doubt overall.

Extensive experiments have been carried out in order to provide an aqueous pharmaceutical composition which is adequately stable. For example, it has unsuccessfully been attempted to improve the storage stability by adjusting the pH or by adding buffer media.

The object of the present invention was to provide an aqueous composition of 1-(2-dimethylaminoethyl)-3-((2R,4aS,5R,10bS)-5-phenyl-9-trifluoromethyl-3,4,4a,5,6,10b-hexahydro-2H-pyrano[3,2-c]quinolin-2-ylmethyl)urea which can be administered parenterally and has adequate storage stability. In order to be capable of parenteral administration, the aqueous pharmaceutical composition should in addition not comprise any toxicologically unacceptable adjuvants.

Surprisingly, it has been possible to provide a composition which meets these requirements if, in addition to the active compound, it comprises at least one nonionic surfactant and at least one organic solvent containing at least one hydroxyl group. The present invention therefore relates to an aqueous pharmaceutical composition comprising 1-(2-dimethylaminoethyl)-3-((2R,4aS,5R,10bS)-5-phenyl-9-trifluoromethyl-3,4,4a,5,6,10b-hexahydro-2H-pyrano[3,2-c]quinolin-2-ylmethyl)urea as active compound, at least one nonionic surfactant and at least one organic solvent containing at least one hydroxyl group.

“Aqueous” above and below is taken to mean water or mixtures of water with other solvents, in particular organic solvents. If one or more substances are dissolved in water or in the mixtures of water with other solvents, aqueous solutions are present. If the aqueous solution comprises one or more active compounds and if it is suitable for therapeutic or prophylactic use in humans or animals, an aqueous pharmaceutical composition is present. If the aqueous solution/aqueous pharmaceutical composition comprises organic solvents, the latter are preferably those which are suitable for parenteral use, such as dimethyl sulfoxide (DMSO), but in particular alcohols, such as, for example, ethanol, 1,2-propanediol, glycerol, polyethylene glycols and glycofurol.

If a liquid composition comprises exclusively organic solvents or the organic solvents present, naturally and in undiluted form, already have a certain water content (thus, for example, pure ethanol has a water content of about 4 percent), an aqueous composition is not present. If, however, water or an aqueous solution (for example physiological saline solution) is added to a composition of this type, an aqueous composition in the sense of the present application arises.

Above and below, “water” means the solvent water or a solution with water as solvent, for example a solution of water comprising adjuvants, such as isotonic agents, such as, for example, sodium chloride, sodium phosphates or glucose. An example of a solution with water as the sole solvent is physiological saline solution.

1-(2-Dimethylaminoethyl)-3-((2R,4aS,5R,10bS)-5-phenyl-9-trifluoromethyl-3,4,4a,5,6,10b-hexahydro-2H-pyrano[3,2-c]quinolin-2-ylmethyl)urea may be present in the pharmaceutical composition as the free base or in the form of one of its acid-addition salts, such as, for example, as the hydrobromide, hydrochloride, dihydrochloride, acetate, aspartate, benzoate, citrate, fumarate, glutamate, maleate, methanesulfonate or tartrate. The active compound is preferably present in the form of the free base.

Nonionic surfactants are taken to mean surface-active substances which, in contrast to ionic surfactants, do not dissociate into ionic substances in aqueous solution. As surface-active substances, nonionic surfactants have the characteristic structure of surfactants, i.e. they contain at least one polar (hydrophilic) functional group and at least one hydrophobic moiety. In contrast to ionic surfactants, in which the hydrophilic group only arises in aqueous solution as a consequence of dissociation into ions, nonionic surfactants contain at least one polar nonionic functional group which does not dissociate into ions. The aqueous composition preferably comprises non-ionic surfactants having an HLB value >8. HLB is the abbreviation for hydrophilic-lipophilic balance (according to Griffin). The HLB value describes the hydrophilic/lipophilic (amphiphilic) properties of nonionic surfactants and is a dimensionless numerical value between 1 and 20 which is calculated from the ratio of the molecular weights of the lipophilic and hydrophilic fractions in the molecule. A hydrophilic fraction of 100% here corresponds to the value 20.

The aqueous pharmaceutical composition according to the invention comprises one or more nonionic surfactants which are readily tolerated toxicologically, in particular those which can also be administered parenterally. These are, in particular, nonionic surfactants which contain hydroxyl groups and/or ether bridges as hydrophilic functional groups. Examples of nonionic surfactants of this type are polyoxyethylene sorbitan fatty acid esters, polyoxyethylene-polyoxypropylene copolymers (poloxamers), polyethylene glycol fatty acid esters and polyethylene glycol hydroxyfatty acid esters. Polyoxyethylene sorbitan fatty acid esters (polysorbates) are known, inter alia, under the trade name Tween. Suitable polyethylene sorbitan fatty acid esters are, in particular, polyoxyethylene (20) sorbitan monolaurate, polyoxyethylene (20) sorbitan monopalmitate and polyoxyethylene (20) sorbitan monostearate. Preference is given to polyoxyethylene (20) sorbitan monolaurate (for example Tween 20®) and polyoxyethylene (20) sorbitan monooleate (for example Tween 80®), of which polyoxyethylene (20) sorbitan monooleate is particularly preferred.

Polyoxyethylene-polyoxypropylene copolymers are also known under the INCI name Poloxamer. A particularly preferred polyoxyethylene-polyoxypropylene copolymer is Poloxamer 188 (CAS 9003-11-6, for example Lutrol F68®). A preferred polyethylene glycol hydroxyfatty acid ester is polyethylene glycol (15) hydroxystearate (polyethylene glycol 660 12-hydroxystearate, Solutol HS15®), which is also the subject of a monograph in the European Pharmacopoeia under the title macrogol (15) hydroxystearate, or polyethylene glycol ricinoleate (polyoxyl 35 castor oil, Cremophor EL®). The aqueous pharmaceutical composition may comprise one or more non-ionic surfactants.

According to an advantageous embodiment of the invention, the aqueous pharmaceutical composition according to the invention is characterised in that the nonionic surfactant(s) present is (are) polyethylene sorbitan fatty acid ester(s), polyoxyethylene-polyoxypropylene copolymer(s), polyethylene glycol fatty acid ester(s) and/or polyethylene glycol hydroxyfatty acid ester(s).

According to a preferred embodiment of the invention, the aqueous pharmaceutical composition according to the invention is characterised in that the nonionic surfactant(s) present is (are) polyethylene glycol ricinoleate (for example Cremophor EL®), polyoxyethylene (20) sorbitan monooleate (for example Tween 80®), polyoxyethylene (20) sorbitan monolaurate (for example Tween 20®), polyoxyethylene-polyoxypropylene block copolymers (for example Lutrol F68®) and/or polyethylene glycol (15) hydroxystearate (for example Solutol HS15®).

Organic solvents containing at least one hydroxyl group which can be employed are, in particular, mono- or polyhydric alcohols and/or polyethylene glycols. According to an advantageous embodiment of the invention, the aqueous pharmaceutical composition according to the invention is therefore characterised in that the organic solvent(s) containing at least one hydroxyl group is (are) mono- or polyhydric alcohol(s) and/or polyethylene glycol(s). The aqueous pharmaceutical composition according to the invention preferably comprises, as alcohol, ethanol, propylene glycol, glycerol, glycofurol and/or polyethylene glycol(s), particularly preferably ethanol.

Alcohols and polyethylene glycols which may be present are all alcohols and polyethylene glycols which are miscible with water and form a homogeneous (aqueous) phase together therewith and are suitable from a toxicological point of view, preferably those which are also tolerated parenterally.

Mono- and polyhydric alcohols are taken to mean hydrocarbons in which one or more hydrogen atoms has (have) been replaced by a hydroxyl group (OH group). Examples of mono-, di- and trihydric alcohols are ethanol, propylene glycol (1,2-propanediol) and glycerol (1,2,3-propanetriol) respectively. The aqueous pharmaceutical composition according to the invention preferably comprises, as alcohol, ethanol, propylene glycol, glycerol, glycofurol and/or polyethylene glycol, particularly preferably ethanol.

Polyethylene glycols are polycondensation products of the general formula HO—(CH₂—CH₂-O—)ₙH (where n=3 to 200). Use can be made in the aqueous composition according to the invention of polyethylene glycols having an average molecular weight of 200 to 1500, preferably having an average molecular weight of 200 to 600. Particular preference is given to polyethylene glycol having an average molecular weight of 400.

According to a preferred embodiment of the invention, the aqueous pharmaceutical composition is therefore characterised in that the organic solvent(s) containing at least one hydroxyl group is (are) ethanol, propylene glycol, glycerol, glycofurol and/or polyethylene glycol. According to a particularly preferred embodiment of the invention, the aqueous pharmaceutical composition is characterised in that the organic solvent containing at least one hydroxyl group is ethanol.

According to an advantageous embodiment of the invention, the aqueous pharmaceutical composition is characterised in that it comprises 0.1 to 20% by weight of nonionic surfactant(s) and 0.05 to 20% by weight of at least one organic solvent containing at least one hydroxyl group.

According to a preferred embodiment of the invention, the aqueous pharmaceutical composition is characterised in that it comprises 0.2 to 5% by weight of nonionic surfactant(s) and 0.1 to 5% by weight of at least one organic solvent containing at least one hydroxyl group.

According to a particularly preferred embodiment of the invention, the aqueous pharmaceutical composition is characterised in that it comprises 0.5 to 2% by weight of nonionic surfactant(s) and 0.2 to 2% by weight of at least one organic solvent containing at least one hydroxyl group.

The aqueous pharmaceutical composition particularly preferably comprises polyethylene glycol (15) hydroxystearate (for example Solutol HS15®) as nonionic surfactant and ethanol as organic solvent containing at least one hydroxyl group. A particularly preferred embodiment of the aqueous pharmaceutical composition according to the invention is therefore characterised in that it comprises polyethylene glycol (15) hydroxystearate as non-ionic surfactant and ethanol as organic solvent containing at least one hydroxyl group.

If the aqueous pharmaceutical composition is intended to be administered parenterally, for example intravenously, the composition is preferably isotonic. If it comprises polyhydric alcohols, such as, for example, glycerol, or polyethylene glycols, isotonicity can be achieved by adaptation of the amounts thereof that are present. If polyhydric alcohols or polyethylene glycols are not present or are not present in the amount necessary for establishing sotonicity, an additional isotonic agent, preferably a physiologically tolerated salt, such as, for example, sodium chloride or sodium phosphate, or a physiologically tolerated polyol, such as, for example, glucose or glycerol, may be present in a concentration necessary for establishing isotonicity. “Isotonic” in the present application denotes an aqueous composition which has an osmolality of 250 to 350 mOsmol/kg. Aqueous compositions of this type can be administered directly intravenously or also intraarterially substantially without pain.

Investigations have shown that the active compound in the aqueous pharmaceutical composition is only hydrolysed very slowly. Even on storage at room temperature, the active compound in the aqueous pharmaceutical composition according to the invention is so stable that it can be administered parenterally without problems over at least 24 hours from preparation. If the aqueous solution is stored under cool conditions, for example at refrigerator temperature (2-8° C.), or if it is frozen, the stable time is significantly extended again. Storage of the aqueous composition according to the invention at refrigerator temperature is preferred.

The aqueous pharmaceutical composition according to the invention can be prepared by dissolving the active compound and the said further ingredients in the said solvents. The sequence in which the active compound, surfactant(s) and any further adjuvants are dissolved in the aqueous and/or organic solvent can basically be varied as desired. The preparation can also be carried out by firstly dissolving different substances in different solvents and subsequently combining the resultant solutions with one another.

According to an advantageous embodiment of the invention, the aqueous pharmaceutical composition according to the invention is prepared by dissolving the active compound in at least one organic solvent containing at least one hydroxyl group, in at least one nonionic surfactant or in a mixture of at least one organic solvent containing at least one hydroxyl group and at least one nonionic surfactant, where the above-mentioned solutions may optionally also comprise further adjuvants, and the resultant solutions are subsequently combined and mixed with water if the active compound is dissolved in a mixture of at least one organic solvent containing at least one hydroxyl group and at least one nonionic surfactant, with at least one non-ionic surfactant and water if the active compound is dissolved in at least one organic solvent containing at least one hydroxyl group, and with at least one organic solvent containing at least one hydroxyl group and water if the active compound is dissolved in at least one nonionic surfactant.

If the active compound is initially in the form of a solution in at least one nonionic surfactant or in at least one organic solvent containing at least one hydroxyl group, the respective constituents that are still missing (one or more organic solvents containing at least one hydroxyl group and water or one or more nonionic surfactants and water) can be added successively in any desired sequence or added together as a mixture.

For practical considerations, it is advantageous to add the constituents that are still missing together as a mixture. If the active compound is thus initially in the form of a solution in at least one nonionic surfactant, it is preferred to prepare the aqueous composition according to the invention by combining and mixing it with a mixture comprising at least one organic solvent containing at least one hydroxyl group and water. If the active compound is initially in the form of a solution in at least one organic solvent containing at least one hydroxyl group, it is preferred to prepare the aqueous composition according to the invention by combining and mixing it with a mixture comprising at least one nonionic surfactant and water.

It has been found that the active compound is stable in solution over a long period if it is dissolved in the organic solvent or solvent mixture and/or in the nonionic surfactant or surfactant mixture which is (are) present in the aqueous composition according to the invention, i.e. if it is present as a solution in at least one organic solvent containing at least one hydroxyl group and/or in at least one nonionic surfactant. The long-term stability of the active compound in one or more organic solvent(s) containing at least one hydroxyl group, in one or more nonionic surfactant(s) or in a mixture of one or more organic solvent(s) containing at least one hydroxyl group and one or more nonionic surfactant(s) thus enables the active compound to be stored in a stable manner in these solutions over a long period (storage time >1 year). The aqueous solution according to the invention can then be obtained from a storage-stable solution of this type, i.e. from a solution of the active compound in one or more organic solvent(s) containing at least one hydroxyl group, in one or more nonionic surfactant(s) or in a mixture of one or more organic solvent(s) containing at least one hydroxyl group and one or more nonionic surfactant(s), as described, by combining and mixing it with the respective constituents that are still missing.

Mixing solutions is a simple operation which can also be carried out without problems in the clinic. It is thus possible to store the active compound in a stable manner over long periods as a solution in an organic solvent or solvent mixture and/or in the nonionic surfactant or surfactant mixture and only to prepare the aqueous pharmaceutical composition according to the invention as required just before parenteral administration thereof. Owing to the above-mentioned stability of the aqueous pharmaceutical composition obtained in this way, however, the latter does not have to be administered immediately, but instead can also be stored in the interim without problems until it is administered, for at least one day at room temperature, for at least one week at refrigerator temperature (2-8° C.). It is of course also possible to administer the resultant solution immediately after preparation thereof.

As described above, the composition comprising the active compound is stable over a long period as an organic solution in one or more organic solvent(s) containing at least one hydroxyl group, in one or more nonionic surfactant(s) or in a mixture of one or more solvent(s) containing at least one hydroxyl group and one or more nonionic surfactant(s) and enables the aqueous composition according to the invention to be prepared in a simple manner just before administration thereof. The invention therefore also relates to a liquid pharmaceutical composition which is suitable for the preparation of the aqueous pharmaceutical composition according to the invention, which is characterised in that it comprises at least one organic solvent containing at least one hydroxyl group and/or at least one nonionic surfactant. This liquid pharmaceutical composition can comprise up to 15% by weight of water, preferably less than 5% by weight of water, particularly preferably less than 2% by weight of water, are present. The lowest possible water content is preferred, but a slight water content usually cannot be excluded completely, in particular owing to the water present in the liquid composition from the surfactant employed.

According to a preferred embodiment, the liquid pharmaceutical composition which is suitable for the preparation of the aqueous pharmaceutical composition according to the invention comprises at least one organic solvent containing at least one hydroxyl group and at least one nonionic surfactant.

According to a preferred embodiment of the invention, the anhydrous storage form of the active compound (long-term-stable solution) is characterised in that the organic solvent(s) containing at least one hydroxyl group is (are) ethanol, propylene glycol, glycerol, glycofurol and/or polyethylene glycol, and the nonionic surfactant(s) is (are) polyethylene glycol ricinoleate (for example Cremophor EL®), polyoxyethylene (20) sorbitan monooleate (for example Tween 80®), polyoxyethylene (20) sorbitan monolaurate (for example Tween 20®), polyoxyethylene-polyoxypropylene block copolymers (for example Lutrol F68®) and/or polyethylene glycol (15) hydroxystearate (for example Solutol HS15®).

If organic solvent(s) and nonionic surfactant(s) are present, the liquid pharmaceutical composition comprises 5 to 95% by weight of organic solvent(s) containing at least one hydroxyl group and 5 to 95% by weight of nonionic surfactant(s). The invention therefore relates to a liquid pharmaceutical composition which is characterised in that it comprises 5 to 95% by weight of organic solvent(s) containing at least one hydroxyl group and 5 to 95% by weight of nonionic surfactant(s).

The liquid pharmaceutical composition preferably comprises 30 to 70% by weight of organic solvent(s) containing at least one hydroxyl group and 70 to 30% by weight of nonionic surfactant(s), particularly preferably 45 to 55% by weight of organic solvent(s) containing at least one hydroxyl group and 45 to 55% by weight of nonionic surfactant(s). According to a preferred embodiment of the invention, the liquid pharmaceutical composition is therefore characterised in that it comprises 70 to 30% by weight of organic solvent(s) containing at least one hydroxyl group and 30 to 70% by weight of nonionic surfactant(s). According to a particularly preferred embodiment, the liquid pharmaceutical composition is characterised in that it comprises 45 to 55% by weight of organic solvent(s) containing at least one hydroxyl group and 45 to 55% by weight of nonionic surfactant(s).

According to an advantageous embodiment of the invention, the aqueous pharmaceutical composition is prepared by mixing a liquid composition comprising the active compound, at least one nonionic surfactant and at least one solvent containing at least one hydroxyl group with water.

The invention also relates to a process for the preparation of the aqueous pharmaceutical composition, which is characterised in that the liquid pharmaceutical composition is combined with the respective missing liquid(s), i.e. with water, with water and at least one nonionic surfactant or with water and at least one organic solvent containing at least one hydroxyl group.

If the liquid pharmaceutical composition which comprises the active compound comprises only organic solvent(s) or only nonionic surfactant(s) as liquid constituent, it is preferred to prepare the aqueous pharmaceutical composition according to the invention by in each case combining it with mixtures of the missing liquid constituents, i.e. with a mixture of water and at least one nonionic surfactant or with a mixture of water and at least one organic solvent containing at least one hydroxyl group. The invention therefore also relates to a process for the preparation of an aqueous pharmaceutical composition which is characterised in that the liquid pharmaceutical composition, if it comprises only at least one organic solvent containing at least one hydroxyl group or only at least one nonionic surfactant as liquid constituent, is in each case combined with a mixture of the missing liquid constituents, i.e. with a mixture of water and at least one nonionic surfactant or with a mixture of water and at least one organic solvent containing at least one hydroxyl group.

The aqueous solution according to the invention can be obtained in a particularly simple manner by adding a liquid pharmaceutical composition comprising at least one organic solvent containing at least one hydroxyl group and at least one nonionic surfactant to an isotonic aqueous solution, for example physiological saline solution. If the isotonic aqueous solution is present in an infusion bag, this can advantageously be carried out after drawing the liquid pharmaceutical composition up into a syringe, and subsequently injecting it directly into the isotonic aqueous solution by piercing the bag by means of a cannula. The organic solvent(s) and the nonionic surfactant(s) can of course also be added to the physiological saline solution in the same way as separate liquids, where at least one of these liquids comprises the active compound, but addition thereof as a mixture is preferred.

The liquid pharmaceutical composition and the liquid constituents furthermore necessary for the preparation of the aqueous pharmaceutical composition according to the invention can advantageously be combined to form a pack (kit). The invention therefore also relates to a kit comprising a container containing a liquid pharmaceutical composition which comprises at least one organic solvent containing at least one hydroxyl group and at least one nonionic surfactant, and a container containing water which is suitable for the preparation of the aqueous pharmaceutical composition according to the invention.

The working examples explain the invention without being restricted thereto.

### EXAMPLE 1

### Stability in Various Solvents and Compositions

The stability of the active compound in the various solvents and the compositions according to the invention found is tested in stable-time studies. To this end, the compositions prepared are stored at various temperatures, removed from storage at certain times and investigated using suitable analytical methods. The climatic conditions selected are refrigerator temperature (2-8° C.) and 25° C. with a relative atmospheric humidity (RH) of 60%. Possible instabilities are principally evident in the case of the active compound in the formation of degradation products.

The results of the stability investigations are shown in Table 1.

TABLE 1

| | Conc. | Temp. | Content of active compound [%] | | | | | |
|---|---|---|---|---|---|---|---|---|
| Solvent composition | [mg/ml] | [° C.] | Start | 2 hrs | 6 hrs | 24 hrs | 1 week | 1 month |
| Isot. NaCl | * | 25 | 98.8 | 83.4 | 57.3 | | | |
| Isot. NaCl pH 5.0 | * | 2-8 | 72.7 | | | | | 9.2 |
| (citrate buffer) | | | | | | | | |
| 60% propylene glycol | 5 | 25 | 96.6 | | | | 92.2 | 78.7 |
| in isot. NaCl | | | | | | | | |
| 5% ethanol/5% Solutol | 5 | 25 | 98.2 | | | 97.5 ** | 93.2 | |
| HS15 in isot. NaCl | | | | | | | | |
| 5% ethanol/5% Solutol | 5 | 2-8 | 98.2 | | | 98.4 ** | 98.2 | |
| HS15 in isot. NaCl | | | | | | | | |
| 0.8% EtOH/0.8% | 0.6 | 25 | 109.3 | | 108.4 *** | | | |
| Solutol HS15 in isot. | | | | | | | | |
| NaCl | | | | | | | | |
| 0.5% EtOH/0.5% | 0.5 | 2-8 | 104.7 | | | 103.3 | 103.1 | |
| Solutol HS15 in isot. | | | | | | | | |
| NaCl | | | | | | | | |
| 10% Cremophor EL in | 5 | 25 | 96.7 | | | | | 84.0 |
| isot. NaCl | | | | | | | | |
| 10% Cremophor EL in | 5 | 2-8 | 96.7 | | | | 96.7 | 93.2 |
| isot. NaCl | | | | | | | | |
| 10% Solutol HS15 in | 5 | 25 | 98.4 | | | | 95.2 | 85.4 |
| isot. NaCl | | | | | | | | |
| 10% Solutol HS15 in | 5 | 2-8 | 98.4 | | | | 97.0 | 96.3 |
| isot. NaCl | | | | | | | | |
| 50% EtOH/50% | 50 | 25 | 98.3 | | | | 98.2 | 98.4 |
| Solutol HS15 | | | | | | | | |
| Ethanol | 50 | 25 | 98.3 | | | | 98.2 | 98.3 |
| 1,2-Propanediol | 50 | 25 | 98.3 | | | | 98.3 | 98.5 |
| Solutol HS15 | 50 | 25 | 97.9 | | | | 97.9 | 98.0 |
| Cremophor EL | 10 | 25 | 97.5 | | | | | 95.2 |

* at saturation concentration
** 48 hours
*** 8 hours

Analytical test methods:

The identity, purity and assay of the compositions comprising 1-(2-dimethylaminoethyl)-3-((2R,4aS,5R,10bS)-5-phenyl-9-trifluoromethyl-3,4,4a,5,6,10b-hexahydro-2H-pyrano[3,2-c]quinolin-2-ylmethyl)urea are checked by high-performance liquid chromatography with UV detection using an RP-18 column in the high-pressure gradient system after preparation and during the stability studies. The extraction medium and mobile phase used are buffered acetonitrile/water mixtures.

### Preparation of Liquid Compositions which are Suitable for the Preparation of the Aqueous Composition According to the Invention

### EXAMPLE 2

| | | |
|---|---|---|
| 1-(2-Dimethylaminoethyl)-3-((2R,4aS,5R,10bS)- | 50 | mg/ml |
| 5-phenyl-9-trifluoromethyl-3,4,4a,5,6,10b-hexahydro- | | |
| 2H-pyrano[3,2-c]quinolin-2-ylmethyl)urea | | |
| Ethanol/polyethylene glycol (15) hydroxystearate | 50/50 | (m/m) |

The active compound is initially introduced suspended in some of the ethanol. The molten polyethylene glycol (15) hydroxystearate is subsequently added, and the ethanol—incl. evaporation effects—is made up to the target amount. The formulation is packaged under aseptic conditions.

### EXAMPLE 3

| | | |
|---|---|---|
| 1-(2-Dimethylaminoethyl)-3-((2R,4aS,5R,10bS)- | 10 | mg/ml |
| 5-phenyl-9-trifluoromethyl-3,4,4a,5,6,10b-hexahydro- | | |
| 2H-pyrano[3,2-c]quinolin-2-ylmethyl)urea | | |
| Ethanol/polyethylene glycol (15) hydroxystearate | 50/50 | (m/m) |

The active compound is initially introduced suspended in some of the ethanol. The molten polyethylene glycol (15) hydroxystearate is subsequently added, and the ethanol—incl. evaporation effects—is made up to the target amount.

### EXAMPLE 4

| | | |
|---|---|---|
| 1-(2-Dimethylaminoethyl)-3-((2R,4aS,5R,10bS)- | 100 | mg/ml |
| 5-phenyl-9-triflouromethyl-3,4,4a,5,6,10b-hexahydro- | | |
| 2H-pyrano[3,2-c]quinolin-2-ylmethyl)urea | | |
| Ethanol/polyethylene glycol (15) hydroxystearate | 50/50 | (m/m) |

The active compound is initially introduced suspended in some of the ethanol. The molten polyethylene glycol (15) hydroxystearate is subsequently added, and the ethanol—incl. evaporation effects—is made up to the target amount.

### EXAMPLE 5

| | | |
|---|---|---|
| 1-(2-Dimethylaminoethyl)-3-((2R,4aS,5R,10bS)- | 50 | mg/ml |
| 5-phenyl-9-trifluoromethyl-3,4,4a,5,6,10b- | | |
| hexahydro-2H-pyrano[3,2-c]quinolin-2- | | |
| ylmethyl)urea | | |
| Ethanol/polyethylene glycol (15) hydroxystearate/ | 50/25/25 | (m/m) |
| polyoxyethylene (20) sorbitan monolaurate | | |

The active compound is initially introduced suspended in some of the ethanol. The molten polyethylene glycol (15) hydroxystearate is subsequently added, and the ethanol—incl. evaporation effects—is made up to the target amount.

### EXAMPLE 6

| | | |
|---|---|---|
| 1-(2-Dimethylaminoethyl)-3-((2R,4aS,5R,10bS)- | 50 | mg/ml |
| 5-phenyl-9-trifluoromethyl-3,4,4a,5,6,10b-hexahydro- | | |
| 2H-pyrano[3,2-c]quinolin-2-ylmethyl)urea | | |
| Ethanol/polyoxyethylene (20) sorbitan monolaurate | 50/50 | (m/m) |

The active compound is initially introduced suspended in some of the ethanol. The polyoxyethylene (20) sorbitan monolaurate is subsequently added, and the ethanol—incl. evaporation effects—is made up to the target amount

### EXAMPLE 7

| | | |
|---|---|---|
| 1-(2-Dimethylaminoethyl)-3-((2R,4aS,5R,10bS)- | 50 | mg/ml |
| 5-phenyl-9-trifluoromethyl-3,4,4a,5,6,10b-hexahydro- | | |
| 2H-pyrano[3,2-c]quinolin-2-ylmethyl)urea | | |
| Propylene glycol/ | 50/50 | (m/m) |
| polyoxyethylene (20) sorbitan monolaurate | | |

The active compound is initially introduced suspended in some of the propylene glycol. The polyoxyethylene (20) sorbitan monolaurate is subsequently added, and the propylene glycol is made up to the target amount.

### EXAMPLE 8

| | | |
|---|---|---|
| 1-(2-Dimethylaminoethyl)-3-((2R,4aS,5R,10bS)- | 50 | mg/ml |
| 5-phenyl-9-trifluoromethyl-3,4,4a,5,6,10b-hexahydro- | | |
| 2H-pyrano[3,2-c]quinolin-2-ylmethyl)urea | | |
| Propylene glycol/polyethylene glycol (15) | 50/50 | (m/m) |
| hydroxystearate | | |

The active compound is initially introduced suspended in some of the propylene glycol. The molten polyethylene glycol (15) hydroxystearate is subsequently added, and the propylene glycol is made up to the target amount.

### EXAMPLE 9

| | | |
|---|---|---|
| 1-(2-Dimethylaminoethyl)-3-((2R,4aS,5R,10bS)- | 50 | mg/ml |
| 5-phenyl-9-trifluoromethyl-3,4,4a,5,6,10b- | | |
| hexahydro-2H-pyrano[3,2-c]quinolin-2- | | |
| ylmethyl)urea | | |
| Propylene glycol/ethanol/ | 25/25/50 | (m/m) |
| polyethylene glycol (15) hydroxystearate | | |

The active compound is initially introduced suspended in some of the ethanol. The molten polyethylene glycol (15) hydroxystearate is subsequently added, and the ethanol and propylene glycol is made up to the target amount.

### Preparation of the Aqueous Composition According to the Invention

### EXAMPLE 10

Basic recipe of active compound product solution A:

| | | |
|---|---|---|
| 1-(2-Dimethylaminoethyl)-3-((2R,4aS,5R,10bS)- | 50 | mg/ml |
| 5-phenyl-9-trifluoromethyl-3,4,4a,5,6,10b-hexahydro- | | |
| 2H-pyrano[3,2-c]quinolin-2-ylmethyl)urea | | |
| Ethanol/polyethylene glycol (15) hydroxystearate | 50/50 | (m/m) |

### EXAMPLE 11

### Preparation of a Solution for Infusion Comprising 20 mg of Active Compound in 100 ml of Infusion Solution

Addition of 0.4 ml of the above-mentioned active compound product solution A ino an infusion bag containing 100 ml of 0.9% saline solution and 3 ml of an ethanol/polyethylene glycol (15) hydroxystearate 50/50 (m/m) composition. This composition exhibits stability over the requisite time span (about 0.5% hydrolysis after 4 hours at 25° C.) between preparation and infusion. For comparison: the pure active compound in aqueous solution at room temperature exhibits approximately 45% degradation in a comparable time span.

### EXAMPLE 12

### Preparation of a Solution for Infusion Comprising 100 mg of Active Compound in 100 ml of Infusion Solution

Addition of 2.0 ml of the above-mentioned active compound product solution A into an infusion bag containing 100 ml of 0.9% saline solution and 1.4 ml of an ethanol/polyethylene glycol (15) hydroxystearate 50/50 (m/m) composition.

### EXAMPLE 13

### Preparation of a Solution for Infusion Comprising 170 mg of Active Compound in 100 ml of Infusion Solution

Addition of 3.4 ml of the above-mentioned active compound product solution A in an infusion bag containing 100 ml of 0.9% saline solution. This composition exhibits stability over the requisite time span between preparation and infusion (about 0.3% hydrolysis after 4 hours at 25° C.). For comparison: the pure active compound in aqueous solution at room temperature exhibits approximately 45% degradation in a comparable time span.

### EXAMPLE 14

### Preparation of a Solution for Infusion Comprising 430 mg of Active Compound in 100 ml of Infusion Solution

Addition of 8.6 ml of the above-mentioned active compound product solution A in an infusion bag containing 250 ml of 0.9% saline solution. This composition exhibits stability over the requisite time span between preparation and infusion. For comparison: the pure active compound in aqueous solution at room temperature exhibits approximately 45% degradation in a comparable time span.

1. Aqueous pharmaceutical composition comprising 1-(2-dimethylaminoethyl)-3-42R,4aS,5R,10bS)-5-phenyl-9-trifluoromethyl-3,4,4a,5,6,10b-hexahydro-2H-pyrano[3,2-c]quinolin-2-ylmethyl)urea as active compound, at least one nonionic surfactant and at least one organic solvent containing at least one hydroxyl group.

2. Aqueous pharmaceutical composition according to claim 1, characterised in that the nonionic surfactant(s) has (have) an HLB value >8.

3. Aqueous pharmaceutical composition according to claim 2, characterised in that the nonionic surfactant(s) is (are) polyoxyethylene sorbitan fatty acid ester(s), polyoxyethylene-polyoxypropylene copolymer(s), polyethylene glycol fatty acid ester(s) and/or polyethylene glycol hydroxyfatty acid ester(s).

4. Aqueous pharmaceutical composition according to claim 3, characterised in that the nonionic surfactant(s) is (are) glycerol polyethylene glycol ricinoleate, polyoxyethylene (20) sorbitan monooleate, polyoxyethylene (20) sorbitan monostearate, polyoxyethylene-polyoxypropylene block copolymer and/or polyethylene glycol (15) hydroxystearate.

5. Aqueous pharmaceutical composition according to claim 1, characterised in that the organic solvent(s) containing at least one hydroxyl group is (are) mono- or polyhydric alcohol(s) and/or polyethylene glycol(s).

6. Aqueous pharmaceutical composition according to claim 5, characterised in that the organic solvent(s) containing at least one hydroxyl group is (are) ethanol, propylene glycol, glycerol, glycofurol and/or polyethylene glycol(s).

7. Aqueous pharmaceutical composition according to claims 6, characterised in that the organic solvent present is ethanol.

8. Aqueous pharmaceutical composition according to claim 1, characterised in that it comprises 0.1 to 20% by weight of nonionic surfactant(s) and 0.05 to 20% by weight of at least one organic solvent containing at least one hydroxyl group.

9. Aqueous pharmaceutical composition according to claim 8, characterised in that it comprises 0.2 to 5% by weight of nonionic surfactant(s) and 0.1 to 5% by weight of at least one organic solvent containing at least one hydroxyl group.

10. Aqueous pharmaceutical composition according to claim 9, characterised in that it comprises 0.5 to 2% by weight of nonionic surfactant(s) and 0.2 to 2% by weight of at least one organic solvent containing at least one hydroxyl group.

11. Aqueous pharmaceutical composition according to claim 1, characterised in that the nonionic surfactant present is polyethylene glycol (15) hydroxystearate and the organic solvent containing at least one hydroxyl group present is ethanol.

12. Liquid pharmaceutical composition which is suitable for the preparation of an aqueous pharmaceutical composition according to claim 1, characterised in that it comprises at least one organic solvent containing at least one hydroxyl group and/or at least one nonionic surfactant.

13. Liquid pharmaceutical composition according to claim 12, characterised in that it comprises at least one organic solvent containing at least one hydroxyl group and at least one nonionic surfactant.

14. Liquid pharmaceutical composition according to claim 13, characterised in that it comprises 95 to 5% by weight of organic solvent(s) containing at least one hydroxyl group and 5 to 95% by weight of nonionic surfactant(s).

15. Liquid pharmaceutical composition according to claim 13, characterised in that it comprises 70 to 30% by weight of organic solvent(s) containing at least one hydroxyl group and 30 to 70% by weight of nonionic surfactant(s).

16. Liquid pharmaceutical composition according to claim 13, characterised in that it comprises 45 to 55% by weight of organic solvent(s) containing at least one hydroxyl group and 45 to 55% by weight of nonionic surfactant(s).

17. Process for the preparation of an aqueous pharmaceutical composition, characterised in that the liquid pharmaceutical composition according to claim 12 is combined with the respective missing liquid(s), i.e. with water, with water and at least one nonionic surfactant or with water and at least one organic solvent containing at least one hydroxyl group.

18. Process according to claim 17, characterised in that the liquid pharmaceutical composition, if it comprises only at least one organic solvent containing at least one hydroxyl group or only at least one nonionic surfactant as liquid constituent, is in each case combined with a mixture of the missing liquid constituents, i.e. with a mixture of water and at least one nonionic surfactant or with a mixture of water and at least one organic solvent containing at least one hydroxyl group.

19. Kit comprising a container containing a liquid pharmaceutical composition according to claim 13 and a container containing water which is suitable for the preparation of an aqueous pharmaceutical composition.
